(19) United States
(12) Patent Application Publication
(10) Pub. No.: US 2010/0241058 A1
(43) Pub. Date: Sep. 23, 2010

(54) OCT GUIDED TISSUE ABLATION

(76) Inventors: Syed Yosuf AHMED, Richmond Hill (CA); Colin Michael HASSEY, Barrie (CA)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 12/726,867

(22) Filed: Mar. 18, 2010

Related U.S. Application Data
(60) Provisional application No. 61/161,598, filed on Mar. 19, 2009.

(51) Int. Cl. A61N 5/06; A61M 31/00; A61B 19/00

(52) U.S. Cl. 604/20; 128/899

(57) ABSTRACT

A method and system of ablating tissue under optical coherence tomography guidance including inserting an optical coherence tomography catheter into a patient's vasculature; navigating the catheter to a target site; imaging and mapping target tissue at the target site using the catheter; delivering a light-activated therapeutic agent into the target tissue; and illuminating the light-activated therapeutic agent with light emitted from the catheter, thereby activating the therapeutic agent and ablating the target tissue; establishing coordinates of the target tissue under computer tomography imaging whereby the catheter provides a marker visible under computer tomography imaging for establishing a positional relationship between the catheter and the target tissue, establishing a localized magnetic field in the target tissue on the basis of the coordinates obtained during the computer tomography imaging and where the light-activated therapeutic agent is magnetized and substantially retained within the target tissue by way of the localized magnetic field.

### CROSS-REFERENCE TO RELATED APPLICATION

This application is related to and claims priority to U.S. Provisional Patent Application Ser. No. 61/161598 filed Mar. 19, 2009 entitled OCT GUIDED TISSUE ABLATION, the entirety of which is incorporated herein by reference.

### STATEMENT REGARDING FEDERALLY SPONSORED RESEARCH OR DEVELOPMENT

n/a

### FIELD OF THE INVENTION

The present invention pertains to the field of tissue ablation, and particularly to a tissue ablation methodology that uses OCT for real time guidance and feedback.

### BACKGROUND OF THE INVENTION

Contraction of the heart is controlled by electrical impulses generated at nodes within the heart and transmitted along conductive pathways extending throughout the wall of the heart. Certain conditions interrupt or alter these pathways, resulting in abnormal contraction, reduced cardiac output, and even death. These conditions, referred to as cardiac arrhythmias, can involve abnormal generation or conduction of the electrical impulses. Certain cardiac arrhythmias can be treated by deliberately damaging the tissue along a conduction path that crosses a route of abnormal conduction. The tissue destruction may be performed by surgically cutting the tissue and/or applying energy or chemicals to the tissue to form scar that inhibits the abnormal electrical conduction. For example, in treatment of atrial fibrillation, a type of cardiac arrhythmia, it has been proposed to ablate tissue in a partial or complete loop around a pulmonary vein; within the vein itself near the ostium of the vein; within the ostium; or within the wall of the heart surrounding the ostium.

Such tissue destruction in sensitive areas of the anatomy calls for precision in selecting and treating the problematic regions while refraining from the unwanted destruction of healthy tissue regions. In view of this concern, it would be desirable to perform such ablation using a catheter-based device which can be advanced into the heart through the patient's circulatory system and to provide systems and methods for use thereof which allow the physician to acquire information about anatomical structures of the heart and surrounding tissues prior to ablation or other treatment. Such imaging information can be used in positioning the ablation device.

In addition to imaging or otherwise acquiring positional information regarding a treatment device such as a catheter, it would further be beneficial to limit the exposure or region in which therapy is delivered to ensure that only the desired tissue regions are exposed or otherwise affected by the delivered therapy, while minimizing or altogether eliminating the unwanted destruction or exposure of healthy tissue regions to the destructive or therapeutic agents.

### SUMMARY OF THE INVENTION

The present invention advantageously provides an optical coherence tomography-guided tissue ablation system including: a catheter; an optical coherence tomography device provided on the catheter; a light source operably coupled to the optical coherence tomography device; a control unit operably coupled to the light source; where the Optical Coherence Tomography device provided on the catheter provides illumination for both the acquisition of images of target tissue, and light-activation of a therapeutic agent situated in the target tissue.

An optical coherence tomography-guided tissue ablation system is also provided including an optical coherence tomography catheter suitable for acquisition of images of target tissue; a light source operably coupled to the optical coherence tomography catheter; a control unit operably coupled to the light source, where the Optical Coherence Tomography catheter provides illumination for both the acquisition of images of target tissue, and light-activation of a therapeutic agent situated in the target tissue. The system may include at least one magnetic field module for generating, a localized magnetic field in the target tissue; where the localized magnetic field promotes localization of a magnetized therapeutic agent in the target tissue.

A method of ablating tissue under optical coherence tomography guidance is provided, including inserting an optical coherence tomography catheter into a patient's vasculature; navigating the catheter to a target site; imaging and mapping target tissue at the target site using the catheter; and delivering a light-activated therapeutic agent into the target tissue; illuminating the light-activated therapeutic agent with light emitted from the catheter, thereby activating the therapeutic agent and ablating the target tissue. The method may also include establishing coordinates of the target tissue under computer tomography imaging whereby the catheter provides a marker visible under computer tomography imaging for establishing a positional relationship between the catheter and the target tissue; establishing a localized magnetic field in the target tissue on the basis of the coordinates obtained during the computer tomography imaging; delivering a light-activated magnetized therapeutic agent into the target tissue, the magnetized therapeutic agent being substantially retained within the target tissue by way of the localized magnetic field; illuminating the light-activated magnetized therapeutic agent with light emitted from the catheter; thereby activating the therapeutic agent and ablating the target tissue.

A therapeutic agent localization system is also provided, including at least one magnetic field module provided on a positionable gantry movable about a patient; the at least one magnetic field module being operable to generate a localized magnetic field at a predefined tissue target of the patient, the localized magnetic field promoting localization of a magnetized therapeutic agent in the tissue.

A method of localizing a therapeutic agent at a target tissue being treated is provided, including establishing coordinates of an area to be treated; establishing a localized magnetic field in the target tissue on the basis of the coordinates; delivering a magnetized therapeutic agent into the target tissue, the magnetized therapeutic agent being substantially retained within the target tissue by way of the localized magnetic field.

A composition for magnetic field-facilitated drug delivery includes a carrier particle capable of being manipulated by a magnetic field; and at least one therapeutic agent associated with the carrier particle.

A composition for magnetic field-facilitated drug delivery is also included, having a carrier particle capable of being manipulated by a magnetic field, at least one coating applied to the carrier particle; and at least one therapeutic agent associated with the coating.

A method of manipulating a magnetic fluid-based composition within a patient's body is also provided, including the application of a localized magnetic field within the body, the localized magnetic field being generated from at least one magnetic field module located external to the body.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows an embodiment of an exemplary OCT catheter suitable for use in OCT-guided tissue ablation in accordance with the principles of the present invention;

FIG. 2 is a schematic diagram of an exemplary OCT-guided tissue ablation system;

FIG. 3 is an exemplary procedure for OCT-guided tissue ablation;

FIG. 4a is a schematic representation of an OCT-guided tissue ablation system wherein a localized magnetic field is created in a patient using a transducer and reflection plate;

FIG. 4b is a schematic representation of another OCT-guided tissue ablation system wherein a localized magnetic field is created in a patient using two transducers;

FIG. 4c is a schematic representation of an embodiment wherein a localized magnetic field is created in a patient using two magnetic field modules;

FIG. 5 is a schematic representation of an OCT-guided tissue ablation system wherein a localized magnetic field is created in a patient using a plurality of transducers; and

FIG. 6 is an exemplary procedure for OCT-guided tissue ablation incorporating a localized magnetic field for localizing a magnetic therapeutic agent in the target tissue.

### DETAILED DESCRIPTION OF THE INVENTION

The OCT-guided tissue ablation method described herein is particularly well suited to the treatment of atrial fibrillation, but as will be appreciated, the methodology will find application in a range of procedures in which neutralizing unwanted tissue growth is required (e.g. cancer). The technology generally uses real-time intraluminal OCT guidance in concert with light-activated dyes or cytotoxin to produce lesions in a controlled predetermined 3-dimensional pattern. Through the interaction of the light energy with the dyes and/or cytotoxins, localized heat and/or active cytotoxic components are produced in sufficient quantity to neutralize unwanted target tissue. By careful application of the dye and/or cytotoxin to the target tissue, healthy, non-target surrounding areas remain largely unaffected when illuminated by the light source.

The OCT-guided tissue ablation system provides a procedure for tissue ablation that generally comprises a pre-treatment mapping/surveying step to isolate the target tissue of interest, a dye/cytotoxin dosage step for delivery of the therapeutic component to the target tissue, a treatment step in which the dye/cytotoxin present in the target tissue is activated, and an optional post-treatment survey. During each of these generalized steps, the OCT imaging component of the system allows for real-time imaging of the tissue being treated, as well as control over the process being executed, so as to increase overall precision.

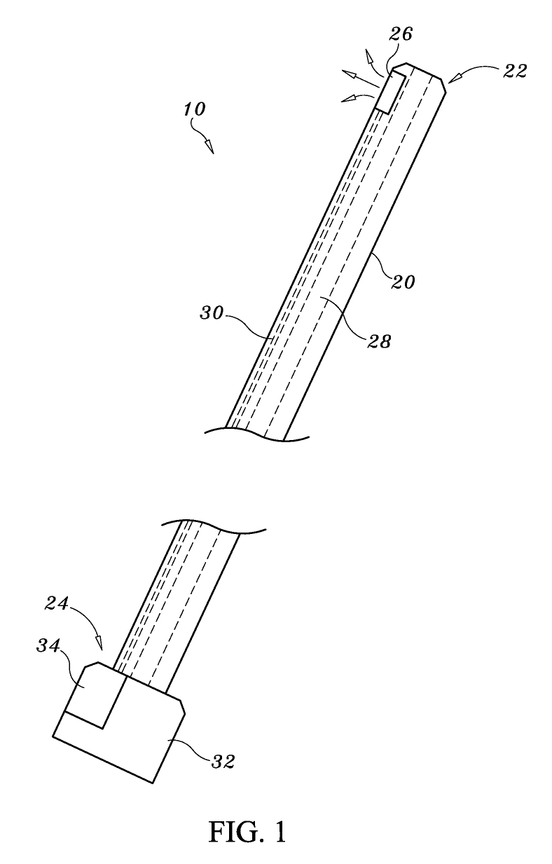

Shown in FIG. 1 is an exemplary OCT catheter 10 suitable for use in OCT-guided tissue ablation. The OCT catheter is comprised of an elongate catheter body 20 having a distal end 22, and a proximal end 24, the catheter being configured generally as an endovascular catheter. The catheter has disposed at the distal end 22 an OCT device 26 suitable for acquisition of images of the target tissue under treatment. Depending on the location of the OCT device 26 relative to the distal end 22, images can be acquired of tissue that is adjacent to the catheter body 20 (e.g. about 90° relative to the longitudinal axis of the catheter), forward of the distal end 22, or areas in between. The elongate catheter body 20 also provides a primary lumen 28, for example suitable for use with a separate guide wire, and a channel 30 for placement of a suitable signal cable (e.g. fiber-optic signal cable, not shown) attached the OCT device 26 situated at the distal end 22 of the catheter body 20. The OCT catheter 10 can be configured with one or more additional channels/Lumens (not shown), for example as a dedicated conduit used to deliver dye/cytotoxin to the tissue under treatment. The proximal end 24 of the catheter 10 is configured with at least one suitable connector 32 to attach the catheter 10 to proximally situated equipment/devices. For example, the proximal end 24 of the catheter can comprise a Luer lock connector to allow attachment to an adaptor, such as a Tuohy borst adaptor. The proximal end 24 is also configured with a connector 34 for attachment of the fiber-optic cable or signal cable to a light source, as described in greater detail below. In some embodiments, where the catheter 10 is used to deliver the dye-cytotoxin, the proximal end can be configured with a connector (not shown) to attach to a suitable dye-cytotoxin reservoir. The elongate catheter body 20 is generally configured to be flexible, but can also be provided as a semi-rigid, or rigid elongate body, as required by the particular implementation of the OCT-guided tissue ablation procedure. The catheter body 20 can be made from a range of materials including, but not limited to silicone rubber, latex and thermoplastic elastomers such as Teflon and other low friction polymers. The catheter body may also be coated with a high lubricity material to reduce friction on passage of the catheter through vessels.

The OCT device 26 provides a three-dimensional histology-like cross-sectional profile of the target tissue. OCT imaging provides an ultra-high level of resolution (up to and exceeding 10 pm), and is capable of providing information relating to the microscope structure of target tissue.

The OCT device 26 is generally provided as an OCT fiber-optic probe provided on or in the vicinity of the distal end 22 of the catheter 10. The device 26 is sufficiently miniaturized so as to be suitable for use in catheters configured for minimally invasive procedures. For example, OCT fiber-optic probes can be as small as 0.014 inches in diameter, thereby reducing any unnecessary bulk to catheter design. While shown as being disposed at the distal end 22 of the catheter, the OCT device 26 can also be located at other points on the catheter 10. For example, in cases where the distal end 22 of the catheter 10 is configured for attachment/deployment of a further medical device, such as a balloon, it may be advantageous to locate the OCT device 26 at a point intermediate between the distal 22 and proximal 24 ends of the catheter body 20,

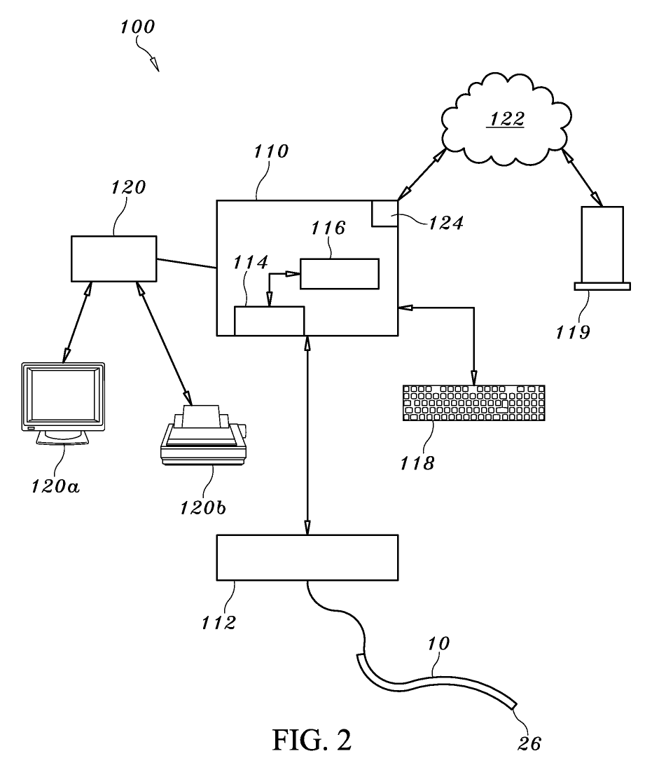

Referring now to FIG. 2, a schematic diagram of an exemplary OCT-guided tissue ablation system 100 is shown. The system generally comprises the control unit 110, a suitable light source 112 operably connected to the control unit 110, and the OCT catheter 10 operably connected to the light source 112. The control unit 110 is generally responsible for data acquisition, imaging processing and general functional control of the OCT catheter 10. The control unit 110 is generally a microcomputer comprised of one or more central processing units 114 connected to volatile memory (e.g. random access memory) and non-volatile memory (e.g. FLASH memory) 116. Data acquisition, image processing and functional control processes are executed in the one or more processing units 114 comprising the control unit. The microcomputer includes a hardware configuration that may comprise one or more input devices 118 in the form of a keyboard, a mouse and the like; as well as one more output devices 120 in the form of a display 120a, printer 120b and the like.

The control unit 110 may also be connected to a core network 122 via a gateway 124, with data acquisition and image processing being based on any suitable server 119 computing environment. While not shown herein, the server 119 may include a hardware configuration that may comprise one or more input devices in the form of a keyboard, a mouse and the like; one or more output devices in the form of a display, printer and the like; a network interface for conducting network communications; all of which are interconnected by a microcomputer comprised of one or more central processing units that itself is connected to volatile memory and nonvolatile memory. The computing environment will also comprise software processes that can be read from and maintained in non-volatile memory (or other computer readable media) that can be executed on the one or more central processing units.

The light source 112 provides light to the OCT device 26 for use in both real time imaging of the target tissue, and activation of the light-activated dye and/or cytotoxin being used. In one embodiment, the light source is a broadband infrared (IR) laser operable at a wavelength in the range of about 1 to about 2 microns. The specific wavelengths used for the tissue ablation methodology are chosen such that absorption and reflection profiles in tissue are minimized, while transmission is maximized. The light source is also chosen to complement/activate the dye or light activated cytotoxin, while also being suitable as the light source for the OCT imaging. As an alternative to the IR laser, other light sources that can be used include a xenon lamp, high intensity LED source, or any other suitable light source capable of producing light in the desired wavelength. In another embodiment, each functionality, that is the real time imaging of the target tissue and the activation of the light-activated dye and/or cytotoxin, may implement separate light sources.

The control unit 110 provides the operator with a real-time image of the tissue under investigation/treatment. From the control unit, the operator is able to view image data, identify and map the target tissue of interest, and plan the dosage of dye-cytotoxin appropriate for the tissue to be treated. The control unit can be configured to be fully automated, wherein the analysis and decision steps are executed independent of the operator, using image analysis and algorithms based on, for example, historical data. The control unit also allows for real time imaging of the administration step in which the determined dosage is delivered to the target tissue of interest. With the dye/cytotoxin in position, continuing under OCT guidance, the target area is illuminated using the light source through the OCT device, thereby activating the dye/cytotoxin. The illumination can be continuous, or periodic, depending on the requirements of the procedure. For example, with tissue that is sensitive to thermal energy, particularly surrounding healthy tissue, the use of periodic illumination whereby the target tissue is illuminated by short powerful bursts of light may be more effective. As tissue is neutralized, the effects of the procedure can be monitored and displayed to the operator in real time, allowing for adjustments and modification of the procedure as necessary to achieve the desired end effect. The control unit also permits the operator the choice of imaging modality, as well as imaging processing to achieve the desired image quality. For example, for obtaining three-dimensional morphology of tissue, either spectral domain OCT or time domain OCT is used. For fluid flow imaging, Doppler OCT is used, while to enhance the contrast of OCT images, time gating is implemented. Image processing as it relates to OCT imaging is generally known and would be implemented here as necessary to achieve the desired resolution and detail necessary to carry out the tissue ablation procedure.

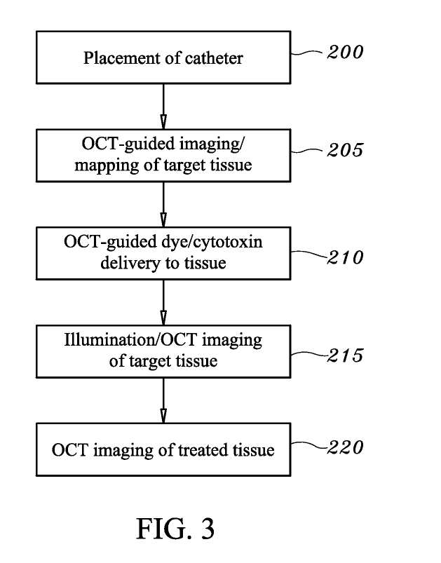

An exemplary procedure for OCT-guided tissue ablation is presented in FIG. 3. In the first step (step 200), the OCT catheter is inserted and directed to the region of interest. The insertion of the OCT catheter may be facilitated by a guide catheter previously inserted into the patient's anatomy. With the OCT catheter located in the general proximity of the target tissue, the OCT catheter is then used to acquire a 3D morphology of the area of interest, surveying for the defective/diseased tissue requiring treatment/ablation (step 205). During this process, the 3D morphology of the area of interest is presented to the operator, for example a doctor, on the display of the control unit. As the OCT catheter is maneuvered within the patient, the images are processed and displayed in real time, enabling the operator to adjust and control the placement of the OCT catheter relative to the target tissue. In the case of atrial fibrillation, the target tissue is generally identified and isolated by monitoring for geometric flutter of the tissue.

With the OCT catheter placed in proximity to the target tissue, the catheter is used to facilitate directed delivery of the appropriate dosage of light-activated dye or cytotoxin (step 210), in accordance with the coordinates determined during initial surveys of the defective/diseased tissue. The OCT device is used in real time to monitor this directed delivery of the therapeutic compound, ensuring its placement in the appropriate tissue. In some embodiments, the absorption of the dye/cytotoxin by the tissue is specifically monitored using Doppler OCT. By monitoring the delivery of the dye/cytotoxin, the operator is able to avoid over-dosing the target tissue, the consequence of which can be the inadvertent delivery of therapeutic compound to the healthy surrounding tissue. Since the dye/cytotoxin are light activated, and given that the light intensity used for OCT imaging is comparatively low with respect to the light required for activation, the tissue receiving the compounds under OCT guidance generally does not react. This allows the operator time to accurately place the compounds where needed, while avoiding placement in healthy tissue.

Once the delivery of the dye/cytotoxin is complete, the OCT catheter is instructed to illuminate the target tissue under treatment (step 215). As such, the OCT device assumes dual functionality wherein in an alternating fashion, the OCT device is operable as an OCT imaging probe, and a light emitting lens for photodynamic therapy, wherein the dye/cytotoxins in the tissue are activated. The dual functionality is provided by the control unit, which appropriately adjusts/modulates the light and collects data in accordance with the timeline that corresponds to the frequency of alternating function of the OCT device. Modulation of the light for each specific function of the OCT device may include adjustment of the power, where increased power is used during light activation of the dye/cytotoxin, and decreased power is used during OCT imaging. The frequency of alteration between operation as an OCT imaging probe and a light emitting lens is adjusted in accordance with permissible limits as defined by the particular dye/cytotoxin in use. In other words, the frequency of alteration is such that when OCT imaging is being done, the dye/cytotoxin is not activated and neutralizing tissue. In some embodiments, the OCT imaging functionality continues under periods of increased light intensity, permitting both activation of the dye/cytotoxin and real-time imaging.

During activation of the dye/cytotoxin, using the OCT imaging, the operator is able to monitor in real time the effect of the procedure on the target tissue. For example, in the case of atrial fibrillation, the desired end effect is the cessation of the cardiac arrhythmia. By monitoring/surveying the target tissue during the course of treatment, including during periods of adjustment and modification of the procedure, the resultant effects con be immediately noted. Since OCT imaging does not expose the patient or doctor with ionizing radiation, the extended use of the imaging technology does not present the same health risks generally associated with CT and x-ray-based imaging. As such, the treatment can be carefully monitored until the desired end effect is noted.

Upon completion of the procedure, the tissue may be subjected to further OCT imaging (step 220) to survey whether or not the particular defected/diseased tissue has been neutralized. The system is configured to store a history of the procedure in memory which is later accessible by a medical practitioner for future reference.

In one embodiment, the aforementioned directed delivery is accomplished through the use of at least one delivery catheter or needle inserted into the primary lumen of the OCT catheter body. The delivery catheter is configured to penetrate the target tissue, allowing for the direct delivery of the dye/cytotoxic substance into the target site. The OCT catheter can also be configured with a specialized channel/lumen to deliver the dye/cytotoxic substance into the general vicinity of the target tissue, such that the dye/cytotoxic compound enters the target tissue through diffusion.

In addition to directed delivery using the OCT catheter, in other embodiments, alternate delivery methodologies can be implemented. For example, delivery may be accomplished through more non-invasive routes, such as, but not limited to oral, topical, transmucosal and inhalation delivery.

In some embodiments, the dye/cytotoxic substance is delivered through direct delivery using an external source (e.g. needle), separate from the OCT catheter. The substance could be injected by a needle from outside the anatomy/tissue (e.g. heart or lumen) undergoing treatment, for example through a second delivery catheter.

In some embodiments, the dye/cytotoxic substance is fed into the bloodstream at another location in the body, with the substance ultimately migrating to the intended target tissue.

In certain tissue types and/or applications, it may be necessary to maintain a relative localization of the dye-cytotoxic substance in the area designated for treatment. A number of methodologies are contemplated for this task.

In one embodiment, the manner of maintaining the dye/cytotoxic substance localized in the area to be treated involves the application of mechanical pressure to the surrounding tissue. In this way, by restricting for example blood flow in the surrounding tissue, the dye/cytotoxic substance delivered into the target area is less likely to dissipate. A non-limiting example of suitable mechanisms for applying pressure include the use of one or more of balloons and clamps.

In another embodiment, the dye/cytotoxic compound could be chemically engineered to either restrict migration from the site of introduction, or engineered to promote travel to a specific tissue type.

In one embodiment, a therapeutic agent localization system may be used to direct and/or contain the dye/cytotoxic substance. For example, the dye/cytotoxic substance may be a component of a magnetic fluid (e.g. ferromagnetic fluid or ferrofluid) that is capable of being directed to a specific target location through the use of an applied localized magnetic field. The fluid could contain either micro- or nano-scale-order particles that are either chemically or physically bonded to the dye/cytotoxin compound, or could be any other type of fluid capable of being manipulated by a magnetic field, for example fluids based on a suspension of magnetically susceptible particles. The fluid could also consist of micro or nano capsules whereby a magnetic or magnetically susceptible particle is encapsulated in a dye/cytotoxic material.

In some embodiments, the aforementioned particles may further comprise one or more coatings to alter or improve their performance in vivo. For example, a coating may be used to render the particle more biocompatible. A coating may be used as a matrix for the incorporation of therapeutic agents. Such drug matrix coatings may be further enabled to provide time release or delayed drug release characteristics. Such coatings may be polymeric or non-polymeric in nature.

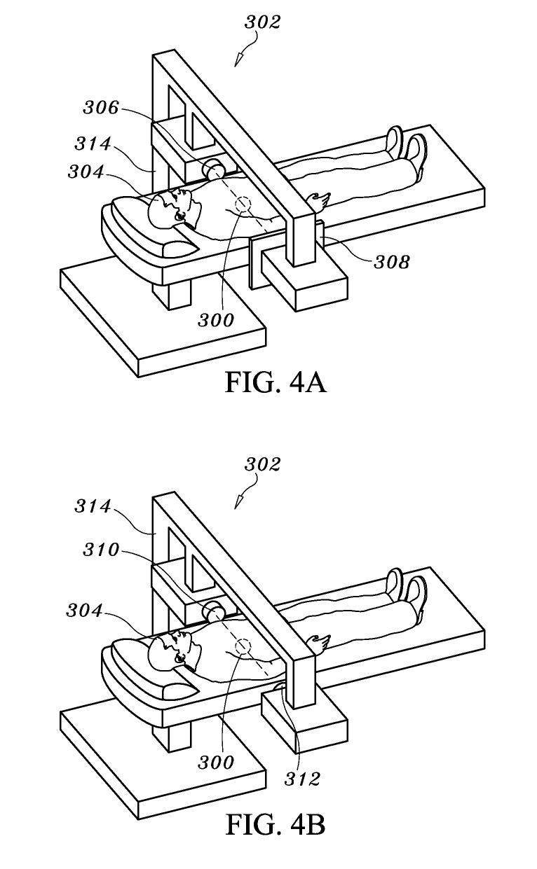

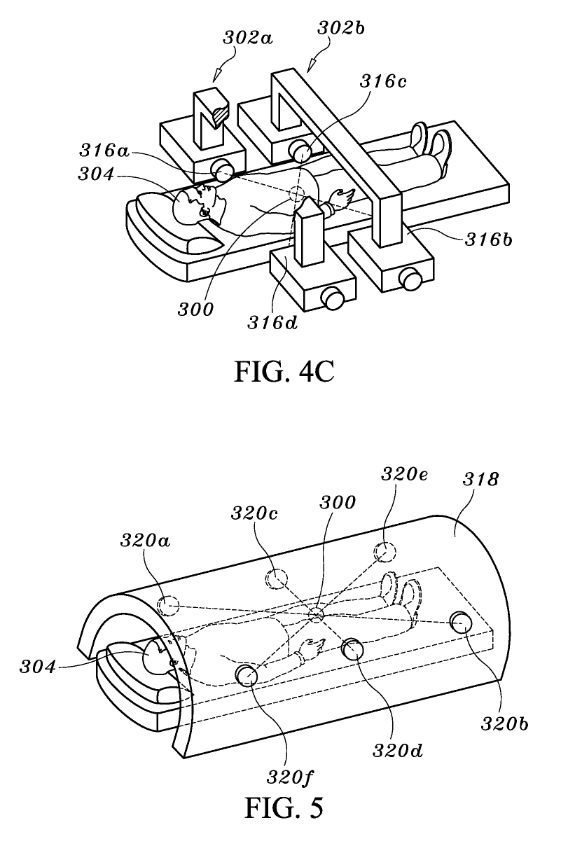

The localized magnetic field may be applied a number of different ways. In one non-limiting example, as shown in FIG. 4a, the localized magnetic field 300 may be applied through the use of a magnetic field module 302 positioned about a patient 304 receiving treatment. In one embodiment, the magnetic field module comprises at least one transducer 306 and at least one opposing reflection plate 308, the at least one transducer and the at least one reflection plate operating cooperatively to create the localized magnetic field 300 at a predetermined location within the patient 304 receiving treatment. Alternatively, as shown in FIG. 4b, the magnetic field module 302 may comprise at least one transducer pair, with each transducer pair comprising a first transducer 310 and an opposing second transducer 312 for creating a localized magnetic field 300 therebetween, in a patient 304 receiving treatment. As will be appreciated, the magnetic field module may be configured a number of different ways, as various arrangements of transducers and reflection plates may be implemented. Regardless of the configuration, to facilitate positioning, the magnetic field module can be mounted on a positionable gantry 314.

As will be appreciated, the magnetic field module 302 serves to create a localized magnetic field 300 in the tissue under treatment, establishing a target zone for magnetic fluids (e.g. the dye/cytotoxic substance) introduced into the body. That is, by way of the local magnetic field 300, the magnetic fluids selectively migrate in accordance with the established field.

Alternatively, magnetic fluids could be introduced using the aforementioned directed delivery, with the localized magnetic field 300 being used to maintain the magnetic fluid within the targeted tissue. This would be particularly advantageous where the magnetic fluid is particularly toxic and diffusion into adjacent tissue should be avoided.

As mentioned above, the localized magnetic field 300 is created using at least one magnetic field module 302 comprising an arrangement of transducers in cooperation with opposing transducers and/or reflection plates positioned about the patient, with the formation of the localized field 300 therebetween. In one embodiment, the magnetic field module is positioned manually, in accordance with a predetermined target as defined by the target tissue to be treated.

In another embodiment, the magnetic field module 302 is provided on an automated positionable gantry 314 capable of movement about a patient 304, as controlled by a processor, for example as provided with the control unit.

In some embodiments, as shown for example in FIG. 4c, the application of a localized magnetic field 300 is accomplished through the use of a plurality of magnetic field modules 302a/302b situated about a patient 304 (portion of magnetic field module 302a removed for clarity). In such an arrangement, the transducers positioned on one side of the patient can be configured to cooperate with any other opposing transducer so as to focus the localized magnetic field at a predetermined target point. For example, transducers 316a and 316b can be configured to act cooperatively, and transducers 316c and 316d can be configured to act cooperatively to generate the localized field 300. The plurality of magnetic field modules may be provided on a positionable gantry (not shown) so as to facilitate movement about a patient. Alternatively, each module in the plurality of magnetic field modules may be mounted on a separate positionable gantry (not shown).

In some embodiments, the plurality of magnetic field modules may be positioned within a field chamber 318. Similar to the embodiment described above, each transducer in the field chamber can be configured to cooperate with an opposing transducer to produce the localized field 300 at a predetermined point. For example, transducer pairs 320a/320b, 320c/320d and 320e/320f can be configured to act cooperatively to generate the localized magnetic field 300 within the patient 304. With this arrangement, with the establishment of the coordinates of the predetermined target, the transducers within the field chamber can be appropriately paired and independently focused to generate the localized field.

In one embodiment, an imaging modality (e.g. Computed Tomography (“CT”)) is used to locate the OCT catheter, and hence the target zone for the localized magnetic field. With the OCT catheter positioned at the target tissue, and by subsequently locating the OCT catheter through CT, the coordinates of the localized magnetic field can be established. In this way, upon delivery of the magnetized dye/cytotoxic substance, the application of the field serves to maintain the substance in the targeted tissue.

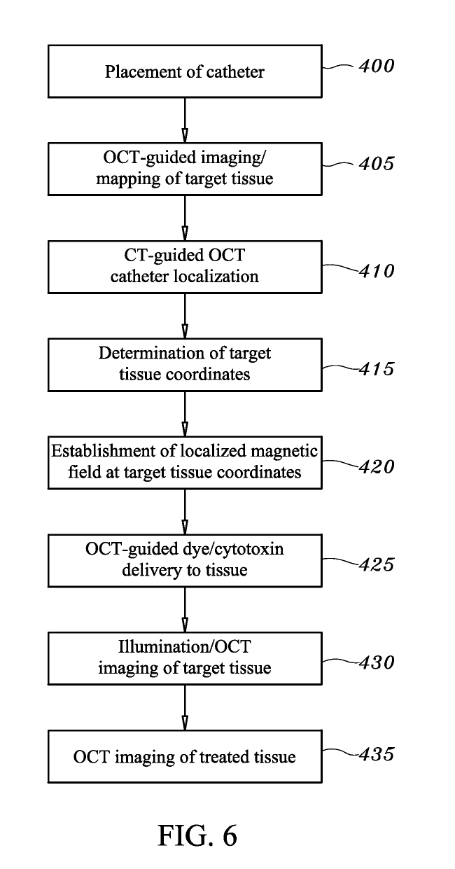

An exemplary procedure of this application is shown in FIG. 6. As generally previously described, in the first step (step 400), the OCT catheter is inserted and directed to the region of interest. The insertion of the OCT catheter may be facilitated by a guide catheter previously inserted into the patient's anatomy. With the OCT catheter located in the general proximity of the target tissue, the OCT catheter is then used to acquire a 3D morphology of the area of interest, surveying for the defective/diseased tissue requiring treatment/ablation (step 405). During this process, the 3D morphology of the area of interest is presented to the operator, for example a doctor, on the display of the control unit. As the OCT catheter is maneuvered within the patient, the images are processed and displayed in real time, enabling the operator to adjust and control the placement of the OCT catheter relative to the target tissue. In the case of atrial fibrillation, the target tissue is generally identified and isolated by monitoring for geometric flutter of the tissue. With the OCT catheter located at the target site, an imaging modality (e.g. CT) is then used (step 410) to locate a marker on the OCT catheter. With a preestablished relationship between the marker and the OCT catheter imaging field, the coordinates of the image field, and hence the target tissue is established (step 415).

Based on the established coordinates, a localized magnetic field is established at the target tissue (step 420), through either manual manipulation of the magnetic field module, or automated positioning through the control of the control unit.

With the OCT catheter placed in proximity to the target tissue, and the localized magnetic field established, the catheter is used to facilitate directed delivery of the appropriate dosage of light-activated dye or cytotoxin, provided in the form of a magnetized fluid. (step 425) to the target tissue. With the application of the localized magnetic field, the magnetized dye/cytotoxic substance is generally maintained in the target tissue, reducing the likelihood that adjacent/surrounding healthy tissue is inadvertently affected. The OCT device is concurrently used in real time to monitor this directed delivery of the therapeutic compound, further ensuring its placement in the appropriate tissue.

Once the delivery of the dye/cytotoxin is complete, the OCT catheter is used to illuminate the target tissue under treatment (step 430), with subsequent OCT imaging (step 435) to survey the results of the treatment procedure.

The above system and procedures have been described using general reference to light activated dyes and light activated cytotoxins. Specific light activated dyes and light activated cytotoxins will now be described, but it should be noted that the following is not intended to be an exhaustive listing. The light activated dyes are generally compounds that absorb light at a specific frequency or range of frequencies and react to produce localized heat that is sufficient to ablate tissue. Similarly, the light activated cytotoxins are generally compounds that absorb light at a specific frequency or range of frequencies and react to chemically alter into a cytotoxic form, capable of ablating tissue. In addition, suitable dyes/cytotoxins are those which are energized/activated at wavelengths where light transmission through tissue is maximized. In this way, surrounding tissues not containing the dye or cytotoxin remain largely unaffected by the procedure. Suitable dyes and cytotoxins are generally known in the field of photodynamic therapy. For example, suitable cytotoxins can be based on a porphyrin platform (e.g. HpD (hematoporphyrin derivative), HpD-based, BPD (benzoporphyrin derivative), ALA {5-aminolevulinic acid), Texaphyrins, or a chlorophyll platform (e.g. Chiorins, Purpurins, Bacteriochlorins). Suitable dyes can be based on Phtalocyanine or Naptholocyanine. It will be appreciated that suitable cytotoxins and dyes may be based on other chemical families and their usage in the presently described technology is contemplated.

It will be appreciated that, although embodiments have been described and illustrated in detail, various modifications and changes may be made. In addition, unless mention was made above to the contrary, it should be noted that all of the accompanying drawings are not to scale. A variety of modifications and variations are possible in light of the above teachings without departing from the scope and spirit of the invention. For example, while a single light source is used in the aforementioned OCT-guided tissue ablation system, the system can alternatively be configured with separate light sources, one for OCT imaging, and one for tissue illumination. While the therapeutic agent localization system was described in respect of dye/cytotoxic substances suitable for use in tissue ablation, the localization system may be used in any therapeutic application in which targeted delivery and/or therapeutic localization is required. Still further alternatives and modifications may occur to those skilled in the art. It will be appreciated by persons skilled in the art that the present invention is not limited to what has been particularly shown and described herein above and is limited only by the following claims.

What is claimed is:

1. An optical coherence tomography-guided tissue ablation system comprising:
a catheter;
an optical coherence tomography device provided on the catheter;
a light source operably coupled to the optical coherence tomography device;
a control unit operably coupled to the light source; wherein the optical coherence tomography device provided provides illumination for acquisition of images of target tissue and light-activation of a therapeutic agent situated in the target tissue.

2. The system of claim 1, further comprising at least one magnetic field module for generating a localized magnetic field in the target tissue.

3. The system of claim 2, wherein the localized magnetic field promotes localization of a magnetized therapeutic agent in the target tissue.

4. A method of ablating tissue under Optical Coherence Tomography guidance comprising:
inserting an optical coherence tomography catheter into a patient's vasculature;
navigating the catheter to a target site;
imaging and mapping target tissue at the target site using the catheter;
delivering a light-activated therapeutic agent into the target tissue; and
illuminating the light-activated therapeutic agent with light emitted from the catheter, thereby activating the therapeutic agent and ablating the target tissue.

5. The method of claim 4, further comprising establishing coordinates of the target tissue under computer tomography imaging whereby the catheter provides a marker visible under computer tomography imaging for establishing a positional relationship between the catheter and the target tissue.

6. The method of claim 5, further comprising establishing a localized magnetic field in the target tissue on the basis of the coordinates obtained during the computer tomography imaging.

7. The method of claim 6, wherein the light-activated therapeutic agent is magnetized and substantially retained within the target tissue by way of the localized magnetic field.

8. A therapeutic agent localization system comprising:
at least one magnetic field module provided on a positionable gantry movable about a patient; the at least one magnetic field module being operable to generate a localized magnetic field at a predefined tissue target of the patient, wherein the localized magnetic field promotes localization of a magnetized therapeutic agent in the tissue.

9. The system of claim 8, wherein the magnetic field module includes at a first magnetic transducer and an opposing second magnetic transducer for creating a localized magnetic field therebetween.

10. The system of claim 8, further comprising a catheter adapted to deliver a magnetized therapeutic agent in the target tissue.

11. A composition for magnetic field-facilitated drug delivery, the composition comprising:
a carrier particle capable of being manipulated by a magnetic field; and
at least one therapeutic agent associated with the carrier particle.

12. The composition of claim 11, further comprising at least one coating applied to the carrier particle; and wherein the at least one therapeutic agent is associated with the coating.
